(19) United States
(12) Patent Application Publication
(10) Pub. No.: US 2025/0375552 A1
(43) Pub. Date: Dec. 11, 2025

(54) USE OF THE EXTRACELLULAR MATRIX TO FORMULATE A FLEXIBLE LUNG BIOADHESIVE

(71) Applicant: TRANSFERTECH SSH S.E.C., Sherbrook (CA)

(72) Inventors: Patrick VERMETTE, Sherbrooke (CA); Laurine GALLIEN, Cepoy (FR); Marco SIROIS, Sherbrooke (CA); Vignesh DHANDAPANI, Sherbrooke (CA); Vickie RINGUETTE, Edmonton (CA)

(21) Appl. No.: 19/148,097

(22) PCT Filed: Jan. 16, 2024
(86) PCT No.: PCT/CA2024/050039

§ 371 Date: Jul. 15, 2025

Related U.S. Application Data
(60) Provisional application No. 63/480,028, filed on Jan. 16, 2023.

(51) Int. Cl. A61L 24/00; A61L 27/36; A61L 27/52

(52) U.S. Cl. CPC A61L 24/0005; A61L 24/0031; A61L 27/3633; A61L 27/52; A61L 2430/22

(57) ABSTRACT

It is provided a bioadhesive comprising an isolated extracellular matrix (ECM) from a decellularized organ with preserved (ultra) structure comprising laminins, collagen 4, collagen-6, collagen-1, fibronectin, elastin, fibrillin, ECM protein 1, proteoglycans, glycoproteins, biglycan, histidine rich glycoprotein, tenascin, and heparan sulfate proteoglycan 2. The bioadhesive described herein and provided composition can be used to seal intra-operative pulmonary air leaks after lung surgery.

### CROSS-REFERENCE TO RELATED APPLICATION

The present application is claiming priority from U.S. Provisional Application No. 63/480,028 filed Jan. 16, 2023, the content of which is hereby incorporated by reference in their entirety.

### TECHNICAL FIELD

It is provided a bioadhesive comprising extracellular matrix particles derived from decellularized organs with preserved (ultra) structure and composition.

### BACKGROUND

Scaffolds made of ExtraCellular Matrix (ECM) have become more and more used in reconstructive surgeries. Each organ and tissue in the body has a distinctive ECM with unique composition and topology. Synthetic or natural materials have been used to create three-dimensional scaffolds to mimic the ECM of organs. This has raised significant interest about the scope of application of ECM-derived scaffolds in tissue engineering and regenerative medicine.

The ECM includes the secretory products of cells in the tissue and organ providing cues aiding in cell proliferation, migration, and differentiation. The ECM is generally composed of a) structural proteins (e.g., collagens, laminins, fibronectin, elastin and tenascins) that are fibrillar and insoluble, able to interact with each other to give a structure and shape to organs, b) globular proteins (e.g., cytokines, growth factors and other matrix metalloproteinases (MMPs)) aiding in cell signalling and ECM network remodelling, and c) proteoglycans (e.g. glycosamino glycans (GAGs)) contributing in hydrating the ECM and helping the interactions with growth factors, cytokines and cell receptors.

One of the most popular approaches to produce ECM is by decellularizing tissues and/or organs. It involves the removal of cellular components of the tissue and hence, only the ECM remains. Different techniques have been used to decellularize organs. Examples include: 1) chemical and enzymatic methods involving the use of detergents such as sodium dodecyl sulfate (SDS), sodium deoxycholate, Triton X-100, 3-([3-cholamidopropyl]dimethylammonio)-1-propanesulfonate hydrate (CHAPS), trypsin, ethylene diaminetetracetic acid (EDTA) and hypertonic solutions and 2) mechanical and physical methods (includes snap-freezing, agitation, freeze-thawing, sonication and hydrostatic pressure). Often, combination of these methods has been reported to be effective in the removal of cellular materials, while preserving the ECM.

Understanding the organization and composition of ECM is necessary considering its potential. Several characterization techniques have been considered. Alcian blue staining has been used as a method to confirm the conservation of GAGs. GAGs such as the hyaluronic acid, chondroitin sulfate, keratan sulfate, dermatan sulfate and heparan sulfate have been used to engineer constructs for a myriad of diseases and for cartilage regeneration.

Collagen is the main constituent of connective tissues, such as the tendons, bones, and skin. In vertebrates, 28 types of collagens (I-XXVIII) have been identified. They occur as triple-helix of α-polypeptide chains. In the ECM, collagens are organized as supramolecular entities defined by the type of collagen composed from different amino-acid sequences and the 3D folding of their tertiary structures. Fibrillar collagens include type I, II, III, V and XI. They merge to form collagen fibers of micrometric sizes and are present in all tissues. Collagen I is the most prominent in the body and is predominantly present as an ECM content in dermis and bone, while it is collagen II for cartilage. Basement membranes (BM) are present in every tissue and are organized glycoproteins that provide a structural and functional support for cells. Collagen type IV is predominant in basement membranes.

Laminins, heterotrimeric glycoproteins that contain α, β and γ polypeptide chains, are associated with collagen IV and are present in BM. They are important in cell attachment, as they help cell integrin receptors to attach to the ECM. 16 different forms of laminins have been identified from 5 α, 3 β and 3 γ chains.

Fibronectin is a dimeric glycoprotein formed by association of two non-identical monomers making two disulfide bridges at the C-terminal. Fibronectin is coded by one gene and is found in the ECMs of most organs, a soluble form also circulates in the blood. It interacts with collagens or integrins.

Tropoelastin monomers, coordinate to form elastin fibers and are associated with fibrillar collagen to impart elasticity to the ECM and compensate for the tensile strength of collagen.

Although, extensive studies have reported the organization and composition of ECM in the native or diseased organ models, very few studies have reported the composition of ECM in decellularized organs.

Lung cancer is very common among both men and women and represents the leading cause of cancer-related deaths worldwide, according to the World Health Organization. Pulmonary resection remains the mainstay of treatment for early-stage lung cancer and sometimes for other benign conditions such as bullous disease.

Prolonged or persistent air leak (PAL) is one of the most common complications following pulmonary resection (wedge, segmentectomy, lobectomy), occurring in up to 30% of the patients.

PAL can be defined as an air leak lasting more than 5 days, according to the Society of Thoracic Surgeons.

An air leak can be assessed postoperatively once a chest tube has been inserted into the pleural space during the operation. Air bubbling through the chest tube into a water-seal chest drainage system indicates an air leak. As long as the air leak occurs, the chest tube cannot be removed because this would likely cause a pneumothorax (abnormal collection of air in the pleural space between the lung and the chest wall). A simple pneumothorax may evolve into a tension pneumothorax, which is a serious life-threatening condition.

Although most of the postoperative air leak resolve spontaneously, PALs may delay chest tube removal causing further complications and increases the length of hospitalization as well as overall costs.

In most cases, during lung surgery, a parenchymal air leak can be easily identified intraoperatively. Patients with severe or moderate emphysema are more prone to having an air leak.

Application of lung sealants during the operation have been shown to provide some control of post-operative air leaks but not to a satisfactory level to justify their routine use.

Most commonly used sealants offer poor adhesion to the lung parenchyma mainly because of lung volume changes during ventilation. Some sealants are just too rigid and lack flexibility and malleability and their application may result in lung parenchymal tear and further exacerbation of the air leak.

An optimal lung bioadhesive with excellent adhesion and malleability properties would be very interesting for thoracic surgeons doing lung operations and as yet to be developed.

There is thus still a need to for new biosealants that can be used for treating air leaks.

### SUMMARY

It is provided an isolated extracellular matrix (ECM) from a decellularized organ comprising laminins, collagen 4, collagen-6, collagen-1, fibronectin, elastin, fibrillin, ECM protein 1, proteoglycans, glycoproteins, biglycan, histidine rich glycoprotein, tenascin, and heparan sulfate proteoglycan 2.

In an embodiment, the isolated ECM further comprises at least one ECM protein, said at least one ECM protein is epidermal growth factor containing fibulin like ECM protein, Fraser ECM complex, and nidogen

In an embodiment, the ECMs maintain their anatomical ultrastructure and have reduced level of hemoglobin.

In a further embodiment, the ECM is formulated as a paste, suspension, or a powder.

In another embodiment, the isolated ECM is freeze-dried and/or stored at 4° C.

It is further provided a bioadhesive composition comprising an isolated extracellular matrix (ECM) as defined herein, a dispersing agent, and water or a phosphate buffered saline solution.

In an embodiment, the dispersing agent is at least one of gelatin, bovine serum albumin, polyethylene glycol, agarose, agar, fibrin, and a combination thereof.

In a further embodiment, the bioadhesive composition described herein further comprises a surfactant.

In an embodiment, the surfactant is at least one of Sodium Dodecyl Sulphate (SDS), CHAPS, Sodium deoxycholate, Triton X-100, or a combination thereof.

In an embodiment, the ECM is in the form of a powder prior to be suspended in the dispersing agent.

In another embodiment, the ECM is from a decellularized organ from a human or an animal.

In an alternate embodiment, the organ is a bladder, liver, pancreas, kidney, or lung.

In a further embodiment, the composition is a gel.

In an embodiment, the composition is sterile.

In a further embodiment, the composition is sterilized by irradiation.

In another embodiment, the isolated ECM particles are suspended heterogeneously in a gelatin hydrogel phase forming a solid suspension.

In an embodiment, the bioadhesive is a cell scaffold.

In a further embodiment, the bioadhesive is a scaffold for pancreatic β-like cells and pancreatic islets.

It is also provided a kit comprising the bioadhesive composition as described herein, and instruction to use same.

In an embodiment, the kit described herein further comprises a delivery device.

In another embodiment, the delivery device is a syringe, spatula and/or swabs.

In a supplemental embodiment, the kit described herein further comprises a crosslinker added in a dried or liquid formulation.

In an embodiment, the crosslinker is coloured to facilitate application.

In a further embodiment, the crosslinker solution is 3% v/v glutaraldehyde.

As provided herewith, the bioadhesive, or the kit described herein can be used to seal air leaks.

In an embodiment, the bioadhesive, or the kit described herein can be used to seal air leaks in lungs after lung surgery, preferably air leaks identified intraoperatively during lung surgery.

It is additionally provided a method of producing an extracellular matrix (ECM) as defined herein from a decellularized organ comprising the steps of providing an organ piece, washing the organ piece in water, freezing the washed organ piece, thawing the frozen organ piece in water, and washing the thawed organ piece in 1×PBS containing 1% penicillin/streptomycin producing the decellularize ECM.

In an embodiment, the organ piece is further immersed in 2M sodium chloride and further washed in water before freezing.

In another embodiment, the washed organ piece is frozen in liquid nitrogen.

In a further embodiment, the washed organ piece is frozen in liquid nitrogen, thawed in water, resubmerged in liquid nitrogen, thawed partially in water, and blended with ice.

In a further embodiment, the blended organ piece is further washed in 2M sodium chloride.

In an embodiment, the thawed frozen organ piece is further washed in ethanol before washing in 1×PBS containing 1% penicillin/streptomycin.

It is also encompassed that the isolated ECM, or the bioadhesive described herein can be used to support cell viability, cell attachment, cell proliferation, and/or cell function.

### BRIEF DESCRIPTION OF THE DRAWINGS

Reference will now be made to the accompanying drawings.

FIG. 1 illustrates a flowchart of a detergent-based and detergent-free process for decellularization of an organ such as a pig bladder in accordance with an embodiment.

FIG. 2 illustrates the result of the process of decellularization of pig bladder in accordance with an embodiment showing in (A) extracellular matrix (ECM) stored following the protocol and in (B) ECM after grinding.

FIG. 3 illustrates in (i) Hematoxylin-Eosin (H&E) staining, Alcian Blue and Nuclear Fast Red (AB&NFR) staining and Scanning Electron Microscopy (SEM) pictures of a) Whole native porcine bladders with excised fat and muscle layers, b) Detergent-free decellularized bladder parts, and c) Detergent-decellularized bladder parts. The scale bars represent 250 μm. +UT indicates urothelium, *LP indicates laminar propria,
MU indicates muscle layer; and in (ii) dsDNA content of native, detergent-free-processed (Det-free), and detergent-processed (Det) bladders. Data presented as mean±standard error.

FIG. 4 illustrates staining with hematoxylin used to stain the nuclei and eosin (E) used to stain the extracellular matrix and cytoplasm on different organs sections before and after decellularization.

FIG. 5 illustrates staining with Alcian Blue, used to stain acidic polysaccharides such as glycosaminoglycans-GAG, and with Nuclear Fast Red (nuclei) on organ sections before and after decellularization.

FIG. 6 illustrates staining with Trichrome Masson, used to stain the collagen (blue), muscle fibres (red), light pink (cytoplasm) and nuclei (brown).

FIG. 7 illustrates dsDNA quantification in native and decellularized organs, providing protein content in the different ECMs.

FIG. 8 illustrates Venn diagrams of the total number of proteins detected in decellularized bladders, kidneys, lungs, livers, and pancreas, whereas the Venn diagrams were prepared using InteractiVenn®.

FIG. 9 illustrates Type IV collagens detected in the decellularized organs: COL4A2, COL4A4, and COL4A5.

FIG. 10 illustrates laminins detected in the decellularized organs: LAM1, LAMA3, LAMB1, LAMB2, LAMB3, LAMC1, and LAMC2.

FIG. 11 illustrates other ECM proteins detected in the decellularized organs: Fibronectin 1 (FN1), ExtraCellular Matrix Protein 1 (ECM1), and Elastin (ELN).

FIG. 12 illustrates in (a) whole native bladder, (b) bladder with the fat and muscular layers excised, (c) diced bladder, (d) native bladder transferred to Erlenmeyer flasks, (e) detergent-based decellularization (15 hours after the process has been initiated), (f) detergent-free decellularized bladder ECM, and (g) detergent-based decellularized bladder ECM.

FIG. 13 illustrates MTT staining of a) INS-1 cells on detergent-free-processed bladders (Det-free); b) Detergent-free-processed bladders with no cells (Det-free Ctrl); c) INS-1 cells on SDS-treated bladders (Det); d) SDS-treated bladders with no cells (Det Ctrl). Scale bar represents 1000 μm. e) Quantification of cells using the CyQUANT™ NF Cell Proliferation Assay. Data are presented as means±standard errors.

FIG. 14 illustrates Hematoxylin and Eosin staining of INS-1 cells cultured for 7 days on the detergent-free decellularized bladder, wherein scale bars represent 50 μm for a, b, and c; 250 μm for d and e. The interactions between the ECM and the cells are indicated by arrows.

FIG. 15 shows the functionality of INS-1 cells validated by a glucose-stimulated insulin secretion (GSIS) assay. S.I. indicates the stimulation index defined as the ratio of insulin concentration secreted at high-glucose to that at low-glucose stimulation. S.I. are represented as means±standard deviations.

FIG. 16 shows in a) β-actin immunostaining of INS-1 cells. Cells cytoskeleton protruding towards the ECM allowing hypothesizing interactions between the cells and the ECM, as highlighted by white stars. b) Representative images of immunostaining of INS-1 cells on detergent-free-processed bladders for insulin and β-actin. Three different samples with 3 images per samples were analyzed. DAPI stained nucleus. Scale bars represent 25 μm.

FIG. 17 shows compositions of bioadhesives tested for air leak on a porcine lung model.

FIG. 18 illustrates pictures of syringes tests containing the bioadhesive described herein showing in (A) a syringe delivered in sterile packaging; in (B) image of the syringe with a long nozzle, containing the bioadhesive; in (C) once heated, the bioadhesive is poured into the syringe; and in (D) image of the syringe with a cut nozzle, containing the bioadhesive.

FIG. 19 illustrates test in a lung with an air leak, showing in (A) preparation of the lung to create an air leak by making a laceration; in (B) application of the bioadhesive; and in (C) validation of the sealing ability of the bioadhesive.

### DETAILED DESCRIPTION

It is provided a bioadhesive derived from a new method to produce extracellular matrix (ECM). This ECM is combined with gelatin and other chemicals and the resulting formulation can be used during thoracic surgery to seal lung parenchyma and thus, avoid postoperative air leak, therefore, allowing faster recovery and less complications for patients.

Accordingly, the isolated extracellular matrix (ECM) described herein is from a decellularized organ comprising, among multiple substances and more than 2700 confirmed proteins for some organs, and not limited to laminins, collagen 4, collagen-6, collagen-1, fibronectin, elastin, fibrillin, ECM protein 1, proteoglycans, glycoproteins, biglycan, histidine rich glycoprotein, tenascin, heparan sulfate proteoglycan 2, epidermal growth factor containing fibulin like ECM protein, Fraser ECM complex, and nidogen. The ECM particles obtained from a decellularized organ as described herein maintain their anatomical ultrastructure and have reduced level of hemoglobin.

Synthetic-derived bioadhesives are known and made solely of synthetic molecules or contain synthetic components. They are formulated with synthetic polymers as a backbone functionalized with reactive groups and are crosslinked or coated via a non-natural cross-linker. The most common synthetic bioadhesives include polyurethane-, polyethylene glycol (PEG)- and cyanoacrylate-based formulations. Biocompatibility has been a concern with some synthetic bioadhesives.

As encompassed herein, it is understood that the term “bioadhesive” also means biosealant. The term “bioadhesion” as used herein refers to the process of adhesion of the composition provided herein containing isolated ECM to a biological surface.

Naturally-derived bioadhesives are extracted purely from biological sources such as human blood, proteins from animal origin (porcine or bovine) or involve an active component (usually crosslinkers like aldehydes), which have been used in combination with animal proteins. A broad classification of naturally-derived bioadhesives include those containing fibrin, chitosan and animal proteins (albumin and gelatin). The primary advantages of natural bioadhesives are their often-good biocompatibility, a degradation resulting into less toxic products and a better elimination from the body. The disadvantages associated with those medical devices are their often-poor mechanical properties, possible variances among batches, degradation rate inside the host and poor adherence to wet tissues.

As encompassed herein, in an embodiment, it is provided a bioadhesive comprising extracellular matrix (ECM) from decellularized organs. ECM was firstly produced from bladder tissue decellularization using SDS as a surfactant (Det). Bladders were first procured from slaughterhouse. They were transported to the lab within 24 hours of animal sacrifice. The muscular and the fatty layers of the bladder were removed. Following delamination, pieces of length and width of approx. 5 mm were cut out using a surgical scissor.

40-50 g of cut bladder was weighed and transferred to Erlenmeyer flasks containing 300 mL of distilled water. Bladders is exemplified but any organ, such as e.g., but not limited to, liver, pancreas, kidney, or lung, is encompassed herein. The bladders were washed two times (2 hours each) in distilled water at room temperature under mechanical agitation at 180 rpm. The decellularization was performed for 30±1 hours in 0.5% Sodium Dodecyl Sulphate (SDS) detergent at room temperature at 180 rpm with addition of fresh SDS thrice in between. Following decellularization, the samples were washed in distilled water for 48 hours at 180 rpm at room temperature with 4 changes of water in-between with the final change (approx. 12 hours) containing 1% Penicillin/Streptomycin. The decellularized bladders were filtered and stored at −20° C. until further use.

As an alternative, it is also encompassed that bladder tissue decellularization can be accomplished using freeze-thawing (cryogenic conditions) (referred to herein a the “freeze-thaw” method or Det-free), osmotic gradients and solvent extraction, in absence of any surfactant. Accordingly, 2M NaCl solution, ethanol 70%, and 1×PBS solutions were prepared. Bladders were delaminated and minced to length and width of 5 mm using surgical scissors. 40-50 g of minced bladders were transferred to Erlenmeyer flasks containing 300 mL of Milli-Q water and incubated 2 times for 2 hours each in Milli-Q water under mechanical stirring (200 rpm) at 37° C. Afterwards, they were incubated for 2 hours at 37° C. and 200 rpm in 2M NaCl and incubated overnight in Milli-Q water at 200 rpm and 37° C.

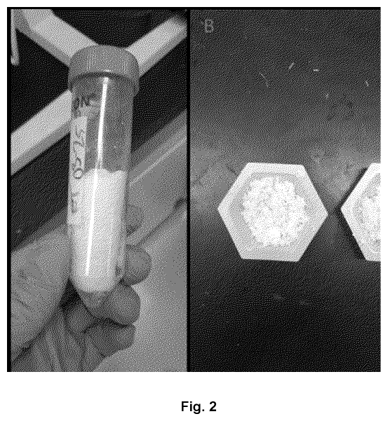

The pieces were submerged in liquid nitrogen for 1 hour making sure the pieces are kept submerged in liquid nitrogen, removed, and incubated for 1 hour in Milli-Q water at 37° C. and 200 rpm. Subsequently, they were submerged in liquid nitrogen again for 1 hour. They were then removed, thawed for 10 minutes in Milli-Q water at 37° C. The pieces were grinded in a blender for 5 minutes with the addition of ice. After grinding, they were incubated for 1 hour in 2M NaCl at 37° C. and 200 rpm, and subsequently for 1 hour each (3 times) followed by overnight in Milli-Q water at 37° C. and 200 rpm. The resulting grinded pieces were rinsed with 70% ethanol, incubated two times (1 h each) in 70% ethanol at room temperature and 200 rpm, and incubated for 1 hour in 1×PBS and then in 1×PBS containing 1% penicillin/streptomycin overnight at room temperature and 200 rpm. Finally, they were filtered, rinsed with 1×PBS, and stored at −20° C. until further use. FIG. 2 shows ECM produced from bladder tissue decellularization using freeze-thaw, osmotic gradients, and solvent extraction.

Two additional protocols were used in addition to the detergent-free methods (i.e., adjustment of pH using ammonium sulfate (Det-free pH) and use of 0.5% EDTA (Det-free EDTA)).

The images of native and decellularized bladders are shown in the FIG. 3 which are the essential component of the bioadhesive described herein. The Hematoxylin-Eosin (H&E) and Alcian Blue-Nuclear Fast Red (AB/NFR) staining of the native bladder (FIG. 3i) revealed the presence of urothelium, as 5-6 layers of transitional epithelial cells. Next to the urothelium, the laminar propria was identified to contain different cell types. The muscularis layer containing the smooth muscle cells have been also identified, densely stained in pink by eosin containing stretched muscle cells. The detrusor muscular layer was not visible, as the process of delamination might have removed it. Following detergent-free decellularization, FIG. 3i shows that nuclei are absent and certain regions of the laminar propria and muscularis are preserved. The detergent-based decellularization also revealed lack of nuclei but, the overall microstructure was not preserved. Alcian blue staining revealed the presence of glycosaminoglycans (GAGs) in decellularized samples produced from both methods. GAGs aid cell attachment and proliferation as well as wound healing.

SEM of native bladder revealed the presence of circular or oval shaped cells attached to a fibrous matrix (FIG. 3i-SEM).

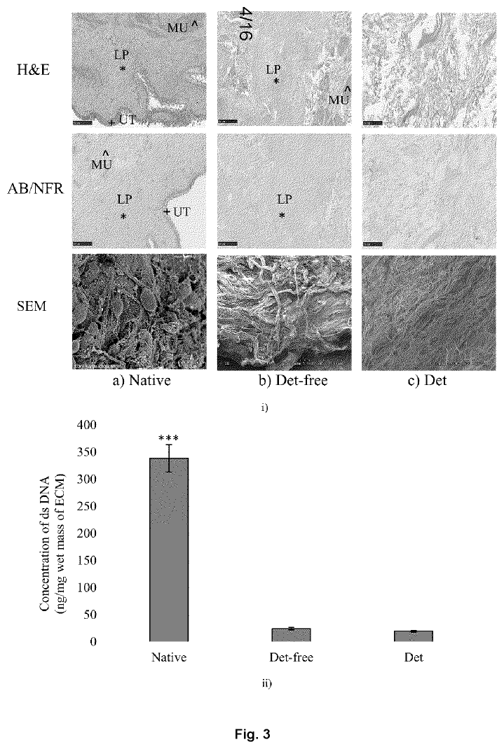

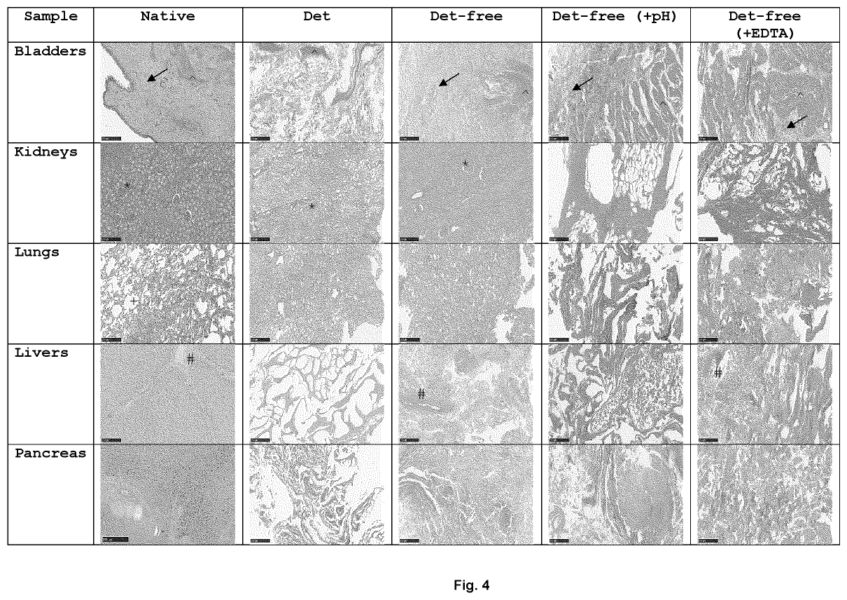

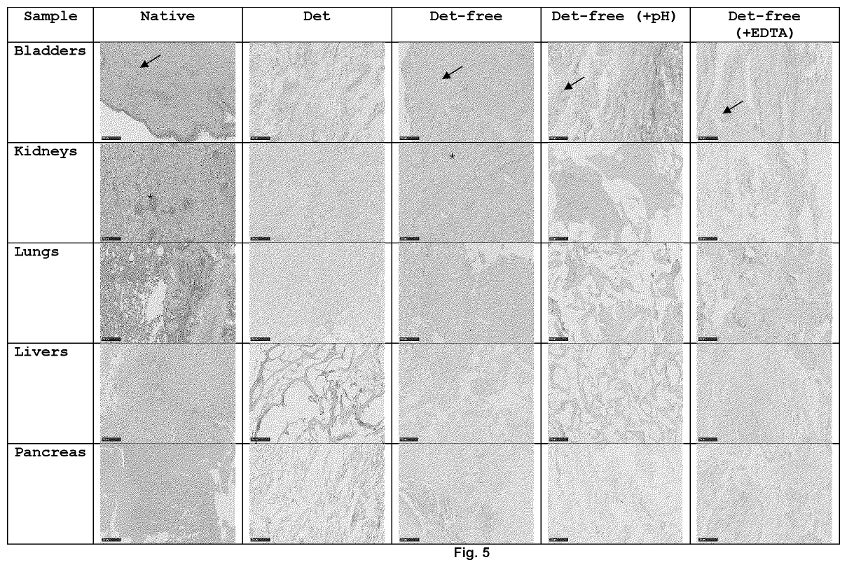

Hematoxylin-Eosin (H&E) and Alcian Blue-Nuclear Fast Red (AB/NFR) staining of native and decellularized porcine organs is showed in FIGS. 4 and 5.

dsDNA quantification in native bladders indicated approximately 20 times the dsDNA concentration than those in decellularized bladder parts, as revealed in FIG. 3ii. The concentration of dsDNA in the detergent-free-treated bladders was comparable to the detergent-treated samples, as no statistical difference was found between the two groups.

Perfusion decellularization has been used to decellularize whole organs allowing retaining the whole architecture of the ECM. The initial step of grinding the organs (except the bladder) was done to increase surface area to facilitate their decellularization. This step enables an efficient decellularization with minimal time of exposure. Since bladder is a muscular tissue, it was diced into smaller pieces with a scissor following delamination instead of grinding. As provided herewith, the process of grinding did not affect the organs, as H&E images of ground organs were comparable to those of the native ones, as shown in FIG. 4.

The glomerulus in kidneys was clearly visible in ground samples. For ground lungs, the bronchioles and alveolar sacs were also visible. Delaminated and diced bladders contained the urothelium and the laminar propria with a certain muscularis. The livers populated with cells along with the septum were identified in ground native samples.

Hemoglobin is a tetramer containing 2 alpha and 2 beta subunits. Following decellularization by the SDS-based and detergent-free methods, samples were analyzed using mass spectrometer for the presence of hemoglobin. The SDS-based method was able to remove cytochrome c and the alpha and beta subunits of hemoglobin, as compared to the detergent-free method (Table 1).

TABLE 1
Detection of alpha and beta subunits of hemoglobin in the decellularized organs.

| Gene names/ | Bladders | | | | Kidneys | | | | Lungs | | | | Livers | | | | Pancreas | | | |
|---|---|---|---|---|---|---|---|---|---|---|---|---|---|---|---|---|---|---|---|---|
| Samples | D | DF | DFP | DFE | D | DF | DFP | DFE | D | DF | DFP | DFE | D | DF | DFP | DFE | D | DF | DFP | DFE |

Hemoglobins

| | | | | | | | | | | | | | | | | | | | | |
|---|---|---|---|---|---|---|---|---|---|---|---|---|---|---|---|---|---|---|---|---|
| Hemoglobin | − | + | + | + | + | + | + | + | + | + | + | + | + | + | + | + | − | − | + | + |
| alpha subunit | | | | | | | | | | | | | | | | | | | | |
| Hemoglobin | − | + | + | + | + | + | + | + | + | + | + | + | + | + | + | + | − | − | + | + |
| beta subunit | | | | | | | | | | | | | | | | | | | | |

D indicates detergent (SDS)-based, DF indicates detergent-free, DFP indicates detergent-free + pH adjustment and DFE indicates detergent-free with EDTA-treatment decellularization.

The release of massive amounts of hemoglobin subunits in the blood plasma have been reported to lead to toxicity in the kidneys. Hence, two additional protocols were developed and applied, based on the detergent-free method. 1) Isoelectric precipitation by adjusting the pH of the suspension to 6.6 using sodium hydroxide and ammonium sulfate during decellularization to precipitate hemoglobin, as the isoelectric point of its β-subunits is 6.1-6.8. 2) Addition of 0.5% EDTA to chelate the iron molecules in hemoglobin. Decellularization using pH adjustment and EDTA treatment resulted in pale brown ECMs for kidneys, livers, and lungs, as compared to dark brown ECMs obtained by the bare detergent-free method not using pH and EDTA treatments. Although mass spectrometry has qualitatively indicated the presence of hemoglobin A and hemoglobin B subunits in the pH-adjusted and EDTA-treated samples, a significant amount of hemoglobin has been removed from the tissues.

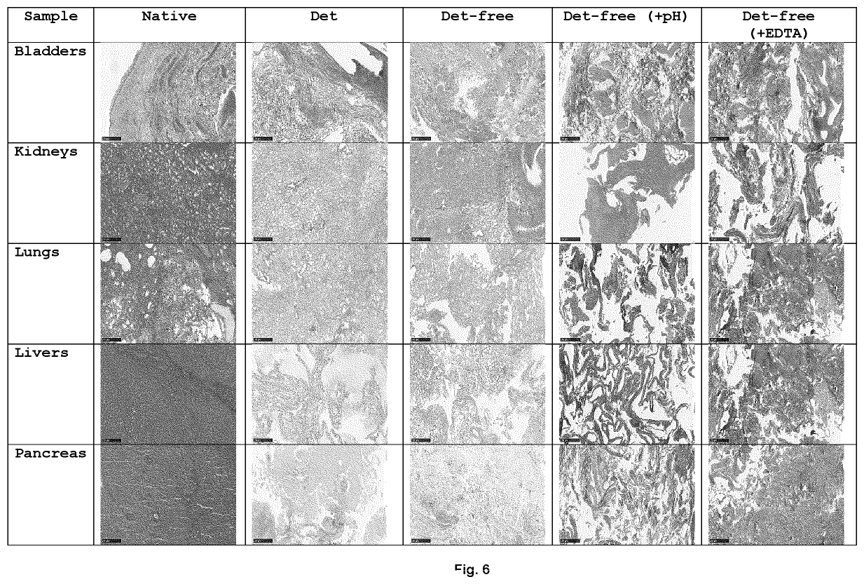

H&E staining of native organs revealed the presence of nuclei (FIG. 4). All tested methods yielded efficient decellularization, as evidence by the lack of nuclei in FIG. 4, compared to the native organs. The detergent-free method was able to preserve certain structures in the organs, such as the laminar propria and the muscularis in bladders, the glomerulus and the distal convoluted tubules in kidneys, bronchioles in lungs and the septum in livers. Detergent (SDS)-based method resulted in matrices in which any structures could be identified. The preservation of GAGs in the decellularized organs was revealed in FIG. 5. In the detergent-free decellularized kidneys and livers, remnant cytoplasm was visible. The Trichrome-Masson staining revealed data provided herewith as shown the presence of collagen fibers in the ECM of native and decellularized organs (FIG. 6).

Accordingly, protocols provided herewith are effective in decellularizing organs (absence of nuclei). Also, the ultrastructure is much better preserved with the “freeze-thaw” method of decellularization. Presence of ECM and GAG organized in a way like that observed in native organs. Trichrome-Masson staining indicated conservation of collagen in the det-free matrices. The “freeze-thaw” method does not involve any chemical agent.

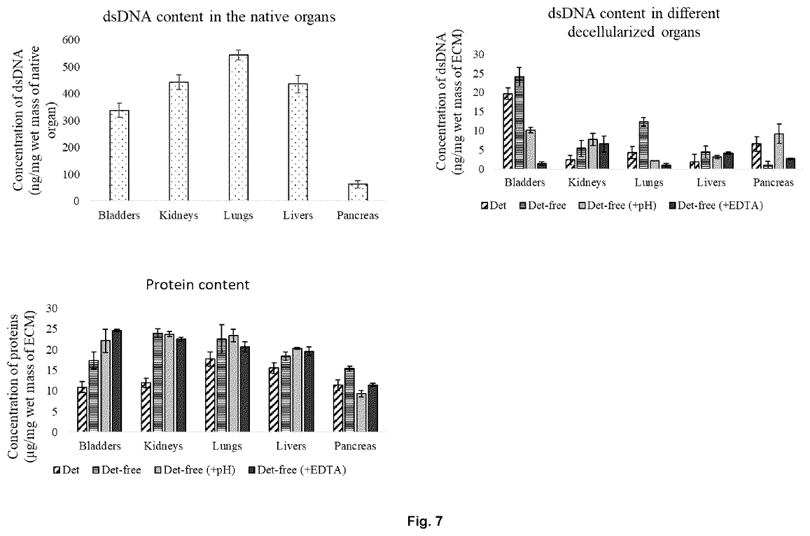

Estimation of dsDNA content in the native and decellularized organs allows assessing the efficiency of nuclear material removal. FIG. 7 illustrates a decreased dsDNA concentration in decellularized organs, as compared to native ones. Quantification of protein content showed a slightly higher protein concentration in the detergent-free methods, as compared to detergent (SDS)-based technique (FIG. 7).

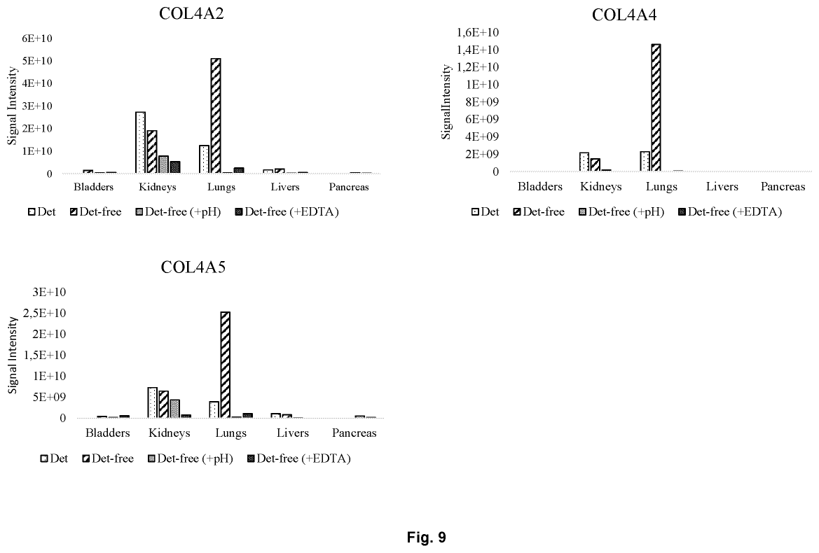

Mass spectrometry of solubilized ECMs have revealed the presence of collagens, laminins, elastin, fibronectin, and other ECM proteins. The proteins of interest collagen IV, elastin, fibronectin, laminins, and others have been screened from mass spectrometric signal intensities and plotted in FIGS. 9-11. The list of other important proteins involved in the formation of the basement membrane, cell adhesion and signaling are shown in Table 2.

TABLE 2
Proteins detected by mass spectrometry.

| Gene names/ | Bladders | | | | Kidneys | | | | Lungs | | | | Livers | | | | Pancreas | | | |
|---|---|---|---|---|---|---|---|---|---|---|---|---|---|---|---|---|---|---|---|---|
| Samples | D | DF | DFP | DFE | D | DF | DFP | DFE | D | DF | DFP | DFE | D | DF | DFP | DFE | D | DF | DFP | DFE |

Collagens

| | | | | | | | | | | | | | | | | | | | | |
|---|---|---|---|---|---|---|---|---|---|---|---|---|---|---|---|---|---|---|---|---|
| COL4A3 | − | − | − | + | + | + | + | + | + | + | + | + | − | − | − | − | − | − | − | − |
| COL4A6 | − | + | + | + | + | + | + | + | + | + | + | + | − | − | − | + | − | − | + | + |
| COL1A1 | + | + | + | + | + | + | + | + | + | + | + | + | + | + | + | + | + | + | + | + |
| COL1A2 | + | + | + | + | + | + | + | + | + | + | + | + | + | + | + | + | + | + | + | + |
| COL6A3 | + | + | + | + | + | + | + | + | + | + | + | + | + | + | + | + | + | + | + | + |
| COL6A1 | + | + | + | + | + | + | + | + | + | + | + | + | + | + | + | + | − | + | + | + |
| COL6A2 | + | + | + | + | + | + | + | + | + | + | + | + | + | + | + | + | + | + | + | + |
| COL6A5 | + | + | + | + | + | + | + | + | + | + | + | + | + | + | + | + | + | − | + | + |
| COL6A6 | + | − | + | + | + | + | + | + | + | + | + | + | + | + | + | + | + | − | + | + |

Glycoproteins

| | | | | | | | | | | | | | | | | | | | | |
|---|---|---|---|---|---|---|---|---|---|---|---|---|---|---|---|---|---|---|---|---|
| FBN1 | + | + | + | + | + | + | + | + | + | + | + | + | + | + | + | + | + | − | + | + |
| FBN2 | − | − | − | − | − | − | − | − | + | − | + | + | + | − | − | − | − | − | + | + |
| BGN | + | + | + | + | + | + | + | + | + | + | + | + | + | + | + | + | − | − | + | − |
| HRG | + | + | + | + | − | + | + | + | + | + | + | + | + | + | + | + | − | − | + | + |
| TNC | − | + | + | + | − | + | + | + | + | + | + | + | + | + | + | + | − | − | + | + |
| HSPG2 | + | + | + | + | + | + | + | + | + | + | + | + | + | + | + | + | + | + | + | + |

Other ECM
Proteins

| | | | | | | | | | | | | | | | | | | | | |
|---|---|---|---|---|---|---|---|---|---|---|---|---|---|---|---|---|---|---|---|---|
| EFEMP1 | + | + | + | + | + | + | + | + | + | + | + | + | + | + | − | + | − | − | − | − |
| EFEMP2 | − | + | + | + | − | + | − | − | − | + | − | + | + | + | − | − | − | − | − | − |
| FRAS1 | − | + | − | − | + | + | + | + | − | − | − | − | − | + | − | − | − | − | − | − |
| NID1 | − | + | + | + | + | + | + | + | + | + | + | + | + | + | + | + | − | − | + | − |
| NID2 | − | + | + | + | + | + | + | + | + | + | + | + | + | + | + | + | − | − | − | − |

D indicates detergent (SDS)-based, DF indicates detergent-free, DFP indicates detergent-free with pH adjustment and DFE indicates detergent-free coupled with EDTA-treatment decellularization. COL: collagen, FBN fibrillin, BGN: Biglycan, HRG: Histidine rich glycoprotein, TNC: Tenascin, HSPG2: Heparan sulfate proteoglycan 2, EFEMP: Epidermal growth factor containing fibulin like ECM protein, FRAS: Fraser ECM complex, NID: Nidogen.

Type IV collagen, laminin-1 and fibronectin are reported to be responsible for β-cell survival and insulin secretion. Protein-coding genes for type IV collagen identified in the decellularized samples include COL4A2, COL4A4 and COL4A5 and their respective signal intensities are shown in FIG. 9. The detergent-free methods were able to conserve type IV collagen in bladders, lungs and livers as compared to the detergent (SDS)-based method. Type IV collagen is the principal component of the basement membrane and is responsible for cell attachment, migration, and proliferation.

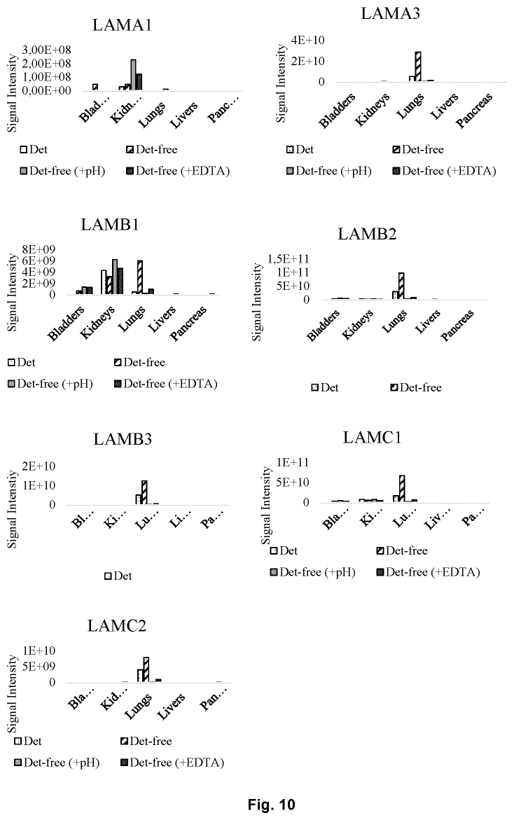

Laminins were found to be more conserved in samples processed using detergent-free method, as compared to those treated using the detergent (SDS)-based technique (FIG. 10). However, decellularized pancreas resulting from all the tested methods lacked laminins. The role of laminins in the basement membrane is to provide a point for cells to attach directly or by entrapping growth factors, as detailed elsewhere.

Other proteins such as fibronectin, elastin, and extracellular matrix protein 1 (ECM1) found in the decellularized organs are illustrated in FIG. 11. The signal intensities of fibronectin and ECM1 in the detergent-free-processed samples indicated a conservation of the respective proteins, as opposed to those treated using the detergent (SDS)-based method. This could be due to the denaturation of the protein due to the SDS treatment.

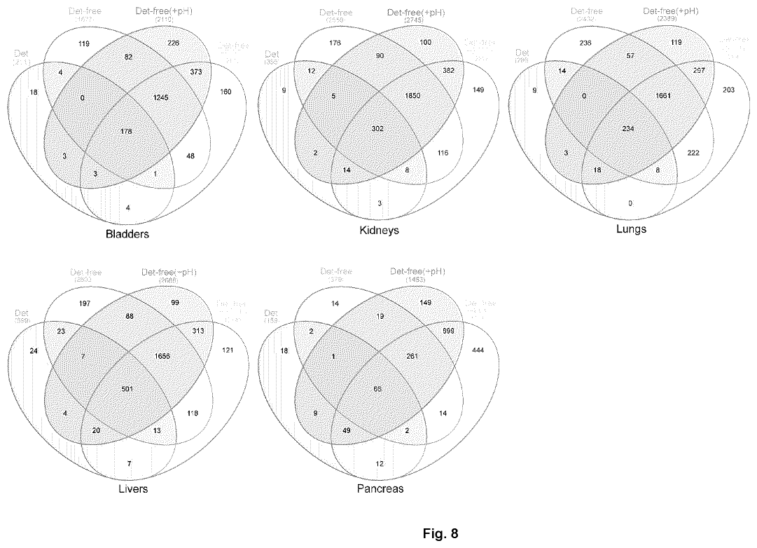

The total numbers of different proteins identified in the five organs decellularized by the four tested methods are shown in FIG. 8. In all cases, the detergent-free methods were able to preserve more proteins, as compared to the detergent (SDS)-based method. Among the detergent-free methods, there was a big overlap of the different proteins preserved except for the pancreas, where the det-free (+pH) and det-free (+EDTA) were able to preserve more proteins compared to the det-free samples. Decellularization using anionic detergents such as CHAPS and 1% SDS has resulted in disruption of the native structure, denaturation of the ECM, and degradation of the basement membrane complex. Decellularization using SDS was found to degrade GAGs, as compared to freeze-thaw method, and using the non-ionic detergent Triton X-100. Intracellular proteins such as the dyneins, tubulins, spectrins, filamin-A and plectins have been detected in the present study by mass spectrometry. Although, multiple studies have confirmed the removal of cytoplasm by histological techniques, very few uses sensitive techniques such as mass spectrometry to identify cytoplasmic proteins in the resulting matrices. Rat lungs have been decellularized using detergents such as SDS, sodium deoxycholate (SDC), and 3-([3-cholamidopropyl]dimethylammonio)-1-propanesulfonate hydrate (CHAPS) and all of them resulted in the retention of cytoplasmic proteins in the matrices.

The pH-adjusted and EDTA-coupled detergent-free decellularization were able to retain certain laminins, and other important proteins such as tenascins, heparan sulfate proteoglycans, biglycans and fibrillin, as compared to the detergent-free decellularization used alone. Cysteine, threonine, and aspartate proteinases are active at an acidic pH and are responsible for the degradation of the intracellular and extracellular proteins.

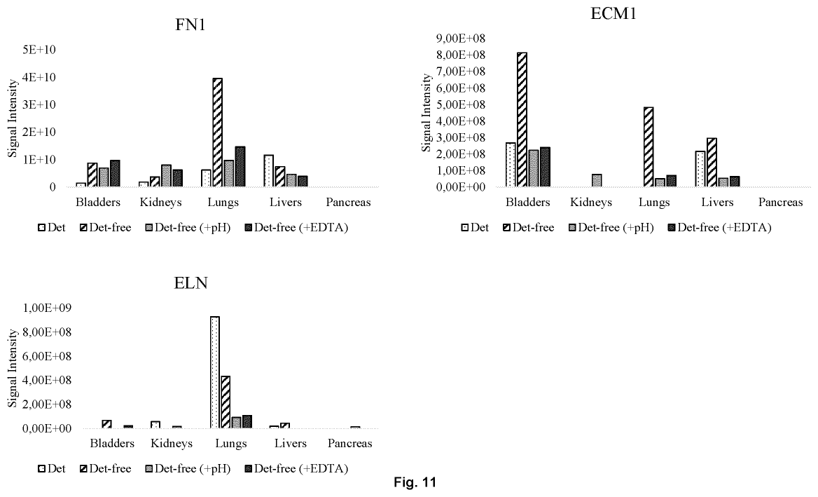

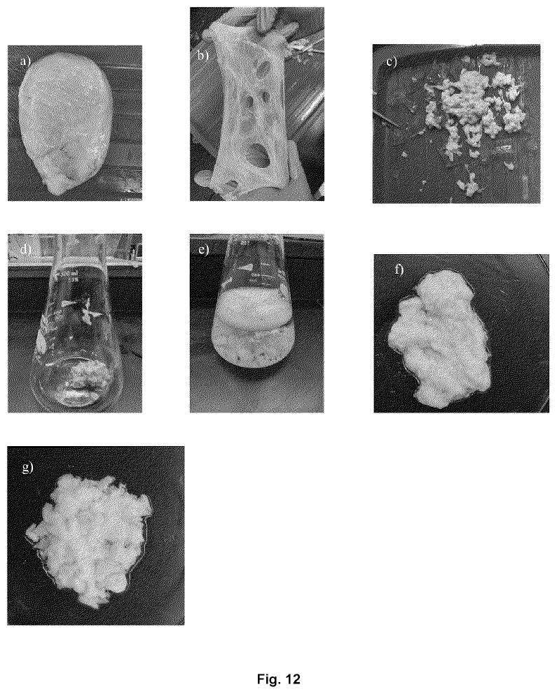

Accordingly, as provided native bladders were cut into smaller pieces after removing fat and muscular layers (see FIG. 12). Dicing the organ into smaller parts and performing decellularization under agitation yields a larger surface area, allowing reducing the duration of the whole process.

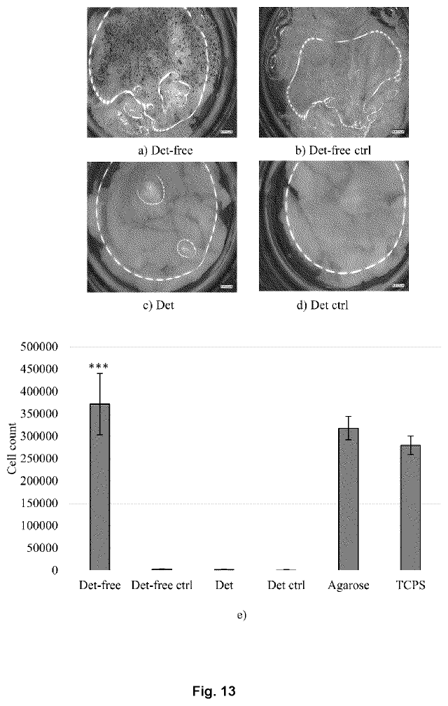

INS-1 cells cultured for 7 days on decellularized bladders were imaged using MTT. Representative images are shown in FIG. 13a, whereas 3 different samples were inspected. Metabolically active cells were observed on detergent-free-processed bladders, as purple-stained cells were visualized (FIG. 13a). Comparing to the controls, this confirms that the crystals did not arise from the matrix itself (FIG. 13b), and that on detergent-processed bladders and control samples, cells were not metabolically active and/or could possibly be dead (FIGS. 13c and 13d).

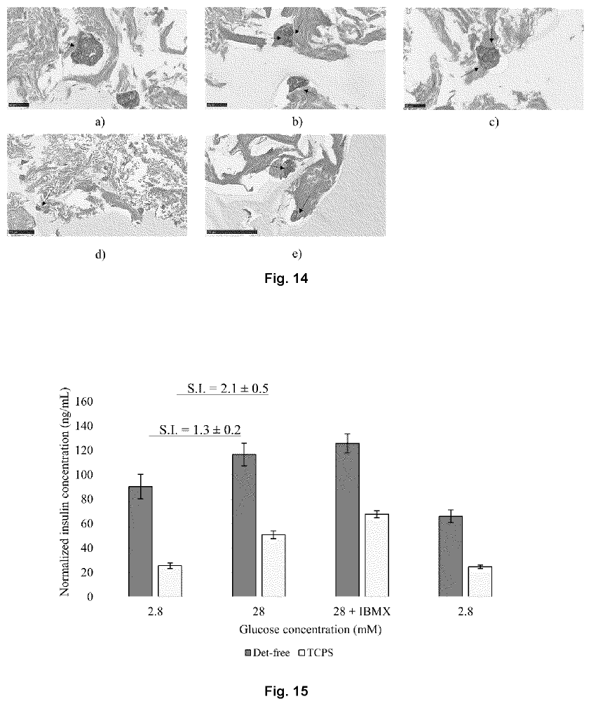

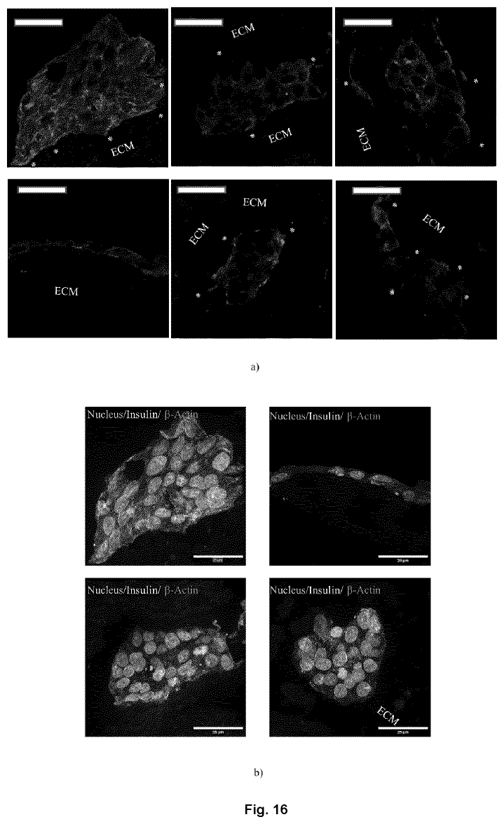

The CyQUANT™ NF Cell Proliferation Assay indicated that INS-1 cells proliferated by approximately 14 times, as compared to the cell number of 25,000 initially seeded on detergent-free-processed bladders. Cell proliferation on tissue culture polystyrene (TCPS) and agarose was comparable to that on detergent-free-processed bladders (FIG. 13e). Detergent-free-processed bladders not seeded with cells (Det-free) revealed that the signal was not remnant from the ECM. Detergent-processed bladders did not support cell proliferation, as there was no significant difference when comparing to the detergent-processed bladders not seeded with cells (Det Ctrl). Better visualization of the interaction between cells and detergent-free-processed bladders was observed by hematoxylin-eosin (H&E) staining of the samples (FIG. 14), revealing that INS-1 cells were interacting with the ECM. Interactions were hypothesized from H&E-stained sections of cells on ECM, as shown in FIG. 14. Cells were firmly attached to ECM parts, as they were resisting media/solution changes and rinsing procedures performed during MTT and CyQUANT™ assays, while this was not the case for agarose i.e., cells formed aggregates in suspension that were not attached to agarose. In addition, immunostaining revealed the shape of cells having extensions pointing towards the ECM, as shown in FIG. 16a. The interactions between the pancreatic β-cells and the matrix proteins such as laminin-1, fibronectin and collagen IV are known to be vital to their survival.

The glucose-stimulated insulin secretion (GSIS) assay revealed that INS-1 cells cultured on detergent-free-processed bladders were functional following a 7-day culture, as shown in FIG. 15. Comparing insulin secretion of INS-1 cells on TCPS, the insulin secretion was higher for the INS-1 cells on detergent-free-processed bladders. The stimulation index i.e., ratio of insulin concentration at high-glucose stimulation to that at low-glucose, was higher for cells on TCPS compared to that on detergent-free-processed bladders. The stimulation index of cells on bladders (1.3±0.2) was comparable to the INS-1 cells cultured in fibrin for 48 hours. The stimulation index of INS-1 cells on TCPS (2.1±0.5) was comparable to the culture on fibronectin-coated plates. The stimulation index according to the insulin secretion in a 3D hydrogel was lower as compared to 2D culture. However, it is difficult to compare the two systems (TCPS and 3D culture), as the 3D culture system can create a diffusional barrier and/or can result in trapped insulin content. The cell-matrix interactions are known to influence the survival and insulin secretion of β-cells by the activation of NF-κB signaling. Laminins are known to be the basement membrane proteins responsible for insulin gene expression and β-cell proliferation. Laminins could have potentially been conserved in the Det-free samples and could have supported insulin expression and thereby functionality (this aspect is the subject of a subsequent study in preparation).

The functionality was also confirmed by immunostaining (FIG. 16b). The intracellular insulin was revealed, confirming that INS-1 cells expressed insulin after the 7-day culture period. The nuclei showed a regular nuclear structure. The staining for β-actin revealed the cells cytoskeleton. The formation and structure of actin along bladder ECM parts, shown in FIG. 16a, revealed that cells were aligned towards the ECM. Certain proteins such as vinculin, talin and integrins are involved in establishing a protein-mediated ECM-actin linkage. Regions of cell attachment to the ECM, also known as the focal adhesion points, could be hypothesized from the observed extensions of the cytoskeleton.

It is thus provided the design of a 3D cell culture system and its validation in the culture of a β-like cell line (INS-1 cells). The system was successfully used to validate the effect of ECM parts on cells in a 3D environment, while preserving as much as possible the ECM (ultra) structure. ECM derived from decellularized porcine bladders can be used as a biocompatible scaffold for the culture of pancreatic β-like cells. Also, it illustrates that the method of decellularization plays a crucial role in cell culture.

The bioadhesive provided can support cell culture and act as a scaffold. As exemplified herein, decellularized organs or ECM provides a favorable micro-environment for the survival, proliferation, and function of e.g., INS-1 cells.

It is further provided a manufacturing process carried out under “aseptic” conditions with the aim of making the manufacturing process robust and “sterile”, which would most likely avoid having to sterilize the material subsequently.

The ECM powder produced is then aliquoted into sterile 50 mL-conical tubes. To determine the sterilization conditions, the microbial load (i.e., bioburden) must first be determined. Four (4) types of bacteria were investigated: 1. Aerobic mesophilic bacteria, 2. Anaerobic mesophilic bacteria, 3. Sporulating aerobic bacteria and 4. Sporulating anaerobic bacteria.

The certificate of analysis revealed that the microbial load was below the quantification capability or detectability threshold of the methods used. In fact, the microbial load was below the threshold of 5 CFU/g. Therefore, the product can be considered essentially sterile. In an alternative, it is also encompassed that sterilization of ECM be performed by gamma irradiation.

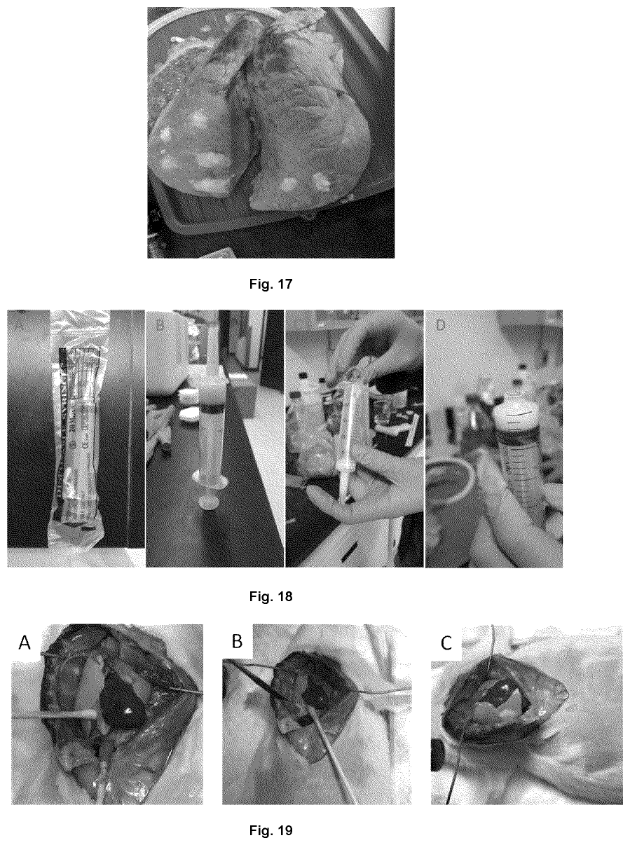

As demonstrated herein, each one of the bioadhesives tested, regardless of the extracellular matrix extraction method or the storage temperature, could adequately seal the air leak after a provoked lesion. After each application of the bioadhesive, water poured on the bioadhesive did not reveal the presence of air bubbles, which means that the air leak was fully closed. In addition, the texture of each one of the samples did not vary according to the storage conditions (see FIG. 17). Samples from the “freeze-thaw” method (Det-free method), however, were somewhat more liquid than those from the “SDS” method, regardless of the storage conditions.

In short, bioadhesives all form a gel with the ability to seal air leaks in the lung. The bioadhesive formulated sterilized (from irradiated ECM) is still functional for sealing air leaks. The bioadhesive can be packaged in a sterilize syringe to be applied.

### Example I

### Protocol to Prepare and Apply the Lung Bioadhesive

5 mL of sterile Milli-Q water was heated in a beaker to 42° C. on a hot stirrer. A magnetic stirrer was introduced. As the first step, bladders were grinded to fine powder using a homogenizer to obtain the ECM.

10% (w/V) porcine gelatin was dissolved in Milli-Q water and autoclaved. 6% (w/v) ECM was weighed, and UV-sterilized. The weighed ECM was added slowly to the stirred gelatin solution at 42° C. The crosslinker solution (3% v/v glutaraldehyde) was applied to the incision on the lung. The prepared ECM solution was added to the incision slowly with a spatula to hold it in place. Gelation was achieved within 6 to 7 minutes.

### Example II

### Porcine Bladder Decellularization and Characterization

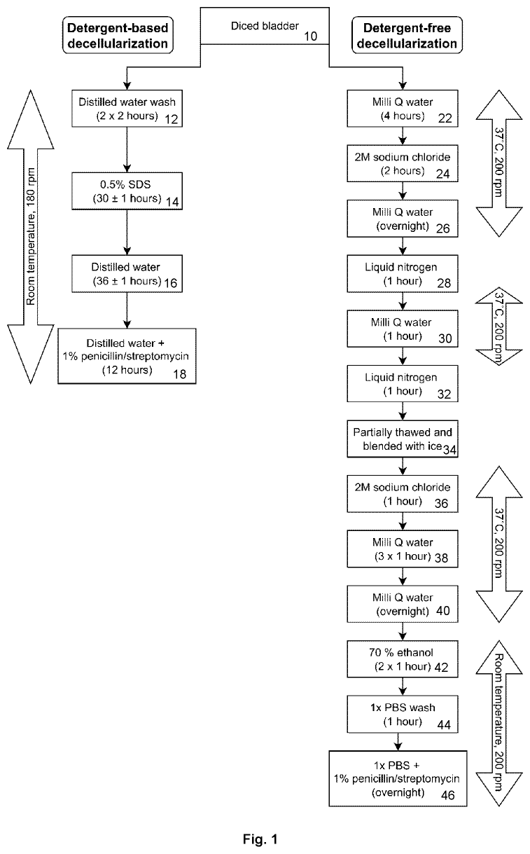

Porcine bladders were freshly procured from the slaughterhouse (Abattoir Regional de Coaticook, Coaticook, Quebec) within 24 hours of animal sacrifice and were placed on ice until use. Fat and muscular layers of the bladder were excised and delaminated using a scissor and a surgical blade. The resulting tissue was diced into approx. 5 mm×5 mm pieces. Decellularization was carried out by two techniques, detergent-based decellularization A or detergent-free decellularization B, as depicted in FIG. 1.

### A—Detergent-Based Decellularization (Det)

The bladder pieces 10 (approx. 40-50 g) were washed with distilled water 12 (300 mL) for 4 hours in a shaker (New Brunswick Innova® 44/44R, Eppendorf, Hamburg, Germany) at 180 rpm with a change of water once. The process of decellularization 14 was carried out using 0.5% Sodium Dodecyl Sulfate (SDS) (161-032, Biorad, Hercules, CA, USA) for 30±1 hours at 180 rpm with the solution replaced thrice during the procedure. The decellularized samples were washed 16 again in distilled water for 36±1 hours at 180 rpm with 3 changes of water in between and the final 12-hour water rinse 18 contained 1% penicillin/streptomycin (15140122, Life Technologies, Carlsbad, CA, USA). The decellularized bladders were filtered and stored at −20° C. until further use.

### B—Detergent-Free Decellularization (Det-Free)

The bladder pieces 10 (approx. 40-50 g) were washed using Milli-Q water 22 (300 mL) for 4 hours in a shaker (Innova® 44/44R, New Brunswick™) at 37° C. and 200 rpm with a change of water once. The process of decellularization 24 was initiated using 2M sodium chloride for an hour followed by an overnight step 26 in Milli-Q water at 37° C. and 200 rpm. The tissues underwent an hour of freezing in liquid nitrogen 28 followed by a thawing step 30 for one hour in Milli-Q water at 37° C. The tissues were again submerged 32 in liquid nitrogen for an hour. Samples were thawed partially 34 in Milli-Q water at 37° C. and were blended (Osterizer 12-speed Blender, Brampton, ON, Canada) with ice, filtered, and washed 36 for one hour in 2M sodium chloride, thrice 38 in Milli-Q water for one hour, and then underwent a final overnight Milli-Q wash 40 at 37° C. and 200 rpm. Samples were washed twice 42 in 70% ethanol, once in 1×PBS (BP665-1, Thermo Fisher Scientific, Waltham, MA, USA) 44 and were subject to an overnight wash 46 in 1×PBS containing 1% penicillin/streptomycin at room temperature and 200 rpm. They were filtered and stored at −20° C. until further use.

C—Detergent-Free Decellularization with pH Adjustment (Det-Free pH)

This third protocol is also based on the second one, except for a modification in the first Milli-Q overnight incubation. The solution was buffered to a pH of 6.6 using sodium hydroxide and ammonium sulfate.

D—Detergent-Free Decellularization with EDTA (Det-Free EDTA)

This fourth protocol is based on the second, except for a modification in the first Milli-Q overnight incubation. Instead of the Milli-Q incubation, the organs were incubated with 0.5% ethylenediamine tetraacetic acid (EDTA).

Bladders (native, detergent-processed, and detergent-free-processed samples) were fixed in 4% paraformaldehyde (PFA) (P16148, Sigma-Aldrich, St-Louis, Missouri, USA) for 48 hours. The samples were processed in a tissue processor, embedded in paraffin, and then sliced into 4-μm thick sections. The sections on glass slides were dried for 48 hours at 37° C. The sections were characterized using 1) Hematoxylin and Eosin, for confirming the absence of nuclei and presence of collagen; 2) Alcian Blue and Nuclear Fast Red staining, for detecting the presence of glycosaminoglycans and nuclei.

The second method to characterize decellularized bladder parts was the SEM to reveal the absence of cells and presence of the 3D fibrous collagen network. Samples were frozen and fixed in 4% PFA. They were washed in PBS twice, fixed in 1% osmium tetroxide and washed twice in distilled water. Dehydration of the samples was achieved by successive ethanol treatments and a final critical point drying in liquid carbon dioxide. The samples were imaged using a Hitachi S-3000N scanning electron microscope after mounting on a stub and coating with gold/palladium.

Samples of native and decellularized bladders were solubilised in proteinase K digestion solution (BP1700, Thermo Fisher Scientific). This step was necessary to eliminate interference of the ECM molecules with the assay and to dissolve the proteins to extract the DNA. The extraction of DNA was performed by the conventional phenol, chloroform and isoamylalcohol (PCI) method (Gilbert et al., 2009, J Surg Res, 152:135-139) and finally precipitated in a mixture of ethanol and sodium acetate. The pelleted DNA was suspended in 1×Tris EDTA buffer. The quantification of double-stranded (ds) DNA was performed using the Quant-iT™ PicoGreen™ dsDNA assay kit (P7589, Thermo Fisher Scientific). The fluorescence was measured at 480 nm excitation (Synergy HT Microplate Reader, Biotek, Agilent, Santa Clara, CA, USA).

### Example III

### Bioadhesive Sterilization

To determine the sterilization conditions, it is necessary to first establish the microbial load (i.e., bioburden). To do this, the analysis was outsourced to a specialized company, Eurofins EnvironeX (Longueuil, Quebec). Four (4) types of bacteria were investigated: 1. Mesophilic aerobic bacteria, 2. Mesophilic anaerobic bacteria, 3. Spore-forming aerobic bacteria, and 4. Spore-forming anaerobic bacteria.

The analysis certificate reveals that the microbial load is below the quantification or detectability threshold of the methods used. In fact, the microbial load is below the threshold of 5 CFU/g.

In a separate step, the ExtraCellular Matrix (ECM) and gelatin were subjected to gamma (Y) irradiation at two different dosages, 10 KGy and 20 KGy, in accordance with FDA regulatory guidelines for medical devices.

The certificate of analysis for this second test reveals that the microbial load for the irradiated products was also below the quantification or detectability threshold of the methods used. In fact, the microbial load was below the threshold of 5 CFU/g.

The results from microbial load studies indicated undetectable levels of microbial load. To ensure the robustness of a biomedical device, the components were subjected to sterilization protocols commonly used for medical devices and approved by regulatory agencies. Additionally, a second gamma irradiation sterilization test was conducted to determine the effect of adding antibiotics to the product. The sterilization method based on γ irradiation was utilized and employed two different dosages (10 KGy and 20 KGy). The ECM and gelatin subjected to irradiation exhibited a change in color. Furthermore, the material was characterized using mass spectrometry analysis to study any protein degradation. A decrease in the number of proteins was observed as detected by mass spectrometry with an increase in dosage strength. ECM samples exposed to a dose of 10 KGy retained more proteins compared to a dose of 20 KGy. The non-irradiated ECM contained 1245 proteins, 10 KGy contained 1060 proteins, and 20 KGy contained 933 proteins. This indicates a loss of 15% of ECM proteins at the 10 KGy dose and a loss of 25% of ECM proteins at the 20 KGy dose compared to non-irradiated ECM. The bioadhesive formulated from irradiated ECM was functional for sealing air leaks.

### Example IV

### Development of a Prototype Syringe for Application of the Bioadhesive

Among several possible options for the application device, a 20 mL plastic syringe with a nozzle measuring 3.5 cm in length and 4 mm in width, designed to prevent the bioadhesive from sticking to the walls of the nozzle and blocking the outlet, was selected. This syringe was delivered in sterile packaging (FIG. 18). A test was also conducted with a cut nozzle, increasing the width of the opening to 8 mm. The incubation time in a 70° C. water bath required for the bioadhesive in the syringe and packaging to reach a temperature of 37 to 40° C. was measured. The bioadhesive, prepared according to the method described above, was first poured into the syringe, and then placed back into the packaging. After 20 minutes, to allow the bioadhesive to solidify, the entire setup was placed in a 70° C. water bath. The temperature of the bioadhesive was regularly monitored, and the time taken for the temperature to reach 45° C. was noted.

Regarding the incubation time required at 70° C. for the bioadhesive to be usable, after 6 minutes of incubation, the temperature of the bioadhesive reached 45° C. Therefore, 6 minutes are required for the bioadhesive in a syringe within its packaging to be ready for application.

### Example V

### Cytotoxicity Testing of the Bioadhesive

A cell culture test was developed to assess the cytotoxicity and in vitro cellular responses of a bioadhesive.

In brief, a gelatin gel was used in which a hole was created using a biopsy punch. This hole served to apply the bioadhesive, which could stick to the walls of the gelatin, mimicking the walls of a lacerated lung. The primary challenge was to produce a gelatin gel resistant to cell culture conditions.

To simultaneously test cytotoxicity and cell proliferation, a WST1 assay was conducted. The cytotoxicity of the bioadhesive was assessed by comparison with controls, using INS1 cells for these initial experiments. To test cell proliferation, a WST1 assay was performed on day 0 and day 3, comparing the data to demonstrate cellular proliferation. During the initial test development, the low pH and excessive residual glutaraldehyde interfered with cell proliferation. To overcome this challenge, the bioadhesive formulation was modified to maintain physiological cell pH. A 1×PBS solution was used to prepare the bioadhesive and employed 1×PBS to prepare 3% glutaraldehyde. This modified formulation was used in all in vivo and ex vivo animal studies. To remove excess glutaraldehyde, the wells were washed five times for 10 minutes each with cell culture medium while agitating. This eliminated any remaining excess glutaraldehyde on the bioadhesive after cross-linking.

The test was conducted with negative and positive controls to compare with the condition using the bioadhesive. For the negative control, wells with the bioadhesive but without cells were used, adding only medium. This allowed to validate the effect of the bioadhesive solely on the WST1 test and eliminated interference from chemical reagents with the test. INS1 cells cultured on TCPS (Tissue Culture Polystyrene) plates were used as a positive control. Additionally, a negative control (without cells) was employed for the TCPS condition. A 4.2× increase in cell proliferation was observed between day 0 and day 3 for the condition with the bioadhesive. A 1.77× increase in cell proliferation was achieved between day 0 and day 3 in the TCPS condition. This indicates a 2.4× increase in cell proliferation with the bioadhesive compared to the TCPS surface.

The cytotoxicity of the bioadhesive was further assessed by comparison with controls using 3T3 cells for these experiments. The test was based on ISO 10993-5 standard involving an established cell line for biocompatibility and cytotoxicity assessment. To test cell proliferation, a WST1 assay was performed on day 0 and day 3, comparing the data to demonstrate cellular proliferation.

The test was conducted with negative and positive controls for comparison with the bioadhesive. For the negative control, bioadhesive wells were used without cells, adding only medium. This allowed to validate the effect of the bioadhesive solely on the WST1 test and eliminated interference from chemical reagents with the test. 3T3 cells cultured on TCPS (Tissue Culture Polystyrene) plates were used as a positive control. Additionally, a negative control (without cells) was employed for the TCPS condition.

A 2.4× increase in cell proliferation was observed between day 0 and day 3 in the condition with the bioadhesive. A 1.2× increase in cell proliferation was achieved between day 0 and day 3 in the TCPS condition. This increase in cell proliferation with the bioadhesive is consistent with previous experiments conducted with INS1 cells.

### Example VI

### Biosealant Short-Term Functionality Testing to Seal Air Leaks in an Animal Model

First animal test on rats: This initial trial aimed to be acquainted with the technique. Glutaraldehyde was applied using a syringe, followed by the application of the bioadhesive with a stick. Although no sealing test was performed, this experience allowed to adjust the surgical procedure and particularly be familiarized with the ventilation apparatus. Importantly, it helped identify a significant variable during application, which is temperature.

Second animal test on rats: Glutaraldehyde was first applied using a syringe and a catheter to direct the application. Similarly, the bioadhesive mixture was also applied using a syringe and a catheter. The bioadhesive mixture risked clogging the syringe due to the considerable size of the ECM particles. The surgical protocol was adjusted, leading to a sternotomy to reveal the thoracic cavity. This trial highlighted the challenge of locating the application of the binding agent. Glutaraldehyde proved difficult to locate due to its excessively liquid, colorless, and transparent nature.

Third and fourth animal tests: The binding agent was pre-mixed with the bioadhesive and applied using a spatula. However, the rapid gelling made the application very difficult, even impossible, hindering effective application. Additionally, it was not feasible to make a second application in case of incomplete sealing during the first application. Autoclaving one of the products resulted in a loss of its sealing power. With this method, the bioadhesive did not penetrate the incision cavity well. Therefore, a wedge, more representative of surgeries performed in humans, was used. The evaluation of the sealant could not be completed as the lung was manipulated during the required waiting time.

Fifth animal test: Glutaraldehyde was applied using a cotton swab, and the bioadhesive mixture was applied using a metal spatula. This test resulted in a successful trial that sealed the leak at a pressure of 9 cm H₂O.

These experiments depicted in FIG. 19 have refined the protocol for applying the bioadhesive. They have also demonstrated the possibility of sealing leaks in live lungs, even in the presence of bleeding.

While the disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

What is claimed is:

1. An isolated extracellular matrix (ECM) from a decellularized organ comprising laminins, collagen 4, collagen-6, collagen-1, fibronectin, elastin, fibrillin, ECM protein 1, proteoglycans, glycoproteins, biglycan, histidine rich glycoprotein, tenascin, and heparan sulfate proteoglycan 2.

2. The isolated ECM of claim 1, further comprising at least one ECM protein, said at least one ECM protein is epidermal growth factor containing fibulin like ECM protein, Fraser ECM complex, and nidogen.

3. The isolated ECM of claim 1, wherein said ECM maintain their anatomical ultrastructure and have reduced level of hemoglobin.

4. The isolated ECM of claim 1, formulated as a paste, suspension, or a powder.

5. The isolated ECM of claim 1, wherein said isolated ECM is freeze-dried and/or stored at 4° C.

6. A bioadhesive composition comprising:
an isolated extracellular matrix (ECM) as defined in claim 1;
a dispersing agent; and
water or a phosphate buffered saline solution.

7. The bioadhesive composition of claim 6, wherein the dispersing agent is at least one of gelatin, bovine serum albumin, polyethylene glycol, agarose, agar, fibrin, and a combination thereof.

8. The bioadhesive composition of claim 6, further comprising a surfactant.

9. The bioadhesive composition of claim 8, wherein the surfactant is at least one of Sodium Dodecyl Sulphate (SDS), CHAPS, Sodium deoxycholate, Triton X-100, or a combination thereof.

10. The bioadhesive composition of claim 6, wherein the ECM is in the form of a powder prior to be suspended in the dispersing agent.

11. (canceled)

12. The bioadhesive composition of claim 11, wherein the organ is a bladder, liver, pancreas, kidney, or lung.

13. The bioadhesive composition of claim 6, wherein the composition is a gel.

14. (canceled)

15. The bioadhesive composition of claim 14, wherein the composition is sterilized by irradiation.

16. The bioadhesive composition of claim 6, wherein the isolated ECM particles are suspended heterogeneously in a gelatin hydrogel phase forming a solid suspension.

17. The bioadhesive composition of claim 6, wherein the bioadhesive is a cell scaffold.

18. The bioadhesive composition of claim 17, wherein the bioadhesive is a scaffold for pancreatic β-like cells and pancreatic islets.

19. A kit comprising:
the bioadhesive composition of claim 6, and
instruction to use same.

20. The kit of claim 19, further comprising a delivery device.

21. The kit of claim 20, wherein the delivery device is a syringe, spatula and/or swabs.

22-34. (canceled)

35. A method of sealing an intra-operative pulmonary air leak in a lung arising after lung resection comprising applying the bioadhesive of claim 6 to the air leak on the lung.
